(19) United States
(12) Patent Application Publication
(10) Pub. No.: US 2003/0065309 A1
(43) Pub. Date: Apr. 3, 2003

(54) Method of delivering liquid through cerebral spinal pathway

(76) Inventor: James C. Barnitz, Schwenksville, PA (US)

Correspondence Address: ALLEN BLOOM, C/O DECHERT, PRINCETON PIKE CORPORATION CENTER, P.O. BOX 5218, PRINCETON, NJ, 08543-5218, US

(21) Appl. No.: 10/131,572

(22) Filed: Apr. 24, 2002

Related U.S. Application Data
(60) Provisional application No. 60/286,063, filed on Apr. 24, 2001.

(51) Int. Cl. A61K 9/22

(52) U.S. Cl. 604/891.1

(57) ABSTRACT

Provided is a method of delivering a physiologically acceptable liquid into the cerebral spinal pathway at high flow rates: initiating flow into a ventricular catheter, through a cerebral spinal pathway, and out a lumbar outflow catheter with the patient in a supine position and using a first flow rate, wherein a positive first value for an outlet pressure is maintained in plumbing from the lumbar outflow catheter; and increasing flow to a second flow rate greater than the first in conjunction with decreasing the outlet pressure to a second, negative value.

This application claims the priority of U.S. Ser. No. 60/286,063, filed Apr. 24, 2001.

The present invention relates to methods of delivering fluid into the cerebral spinal pathway.

Focal cerebral ischemia, or stroke, is the reduction or loss of blood flow to an area of cerebral tissue, denying the tissue sufficient oxygen and other metabolic resources. Similarly, during Traumatic Brain Injury (TBI) and Spinal Cord Injury (SCI) the tissues are also denied sufficient oxygen and other metabolic resources to carry out normal function or survive. Technology that has been explored by Osterholm has identified the cerebral spinal pathway, a connected system of cerebral ventricles and subarachnoid spaces of the brain and spinal cord, as an alternative pathway for delivering oxygen and nutrients to the tissue potentially affected by stroke. This stratagem has been shown in animal models for stroke to remarkably limit damage caused by focal cerebral ischemia.

The approach operates by placing a ventricular catheter into a lateral cerebral ventricle for use in administering an oxygenated fluorocarbon nutrient emulsion into the cerebral spinal pathway. The oxygenated fluorocarbon nutrient emulsion typically is made up of an emulsified fluorocarbon composition, where the fluorocarbon efficiently dissolves and carries gases such as oxygen and carbon dioxide. The composition typically further contains additional nutrients. A second catheter is placed to allow drainage of fluid in the cerebral spinal pathway as needed in view of the injected fluorocarbon composition.

In seeking to provide satisfactory flow rates through the cerebral spinal pathway, it is important to avoid elevated intracranial pressures. The intracranial pressure target is approximately 25 mmHg or less, with 20 mmHg or less preferred. Consistent with the normal physiology of this tissue, pulsatile pressure spikes in excess of this value are acceptable. Also, extremely low or negative pressures in the cerebral spinal pathway must be avoided to avoid collapses of tissue structures. It has now been discovered that negative height differentials at the outlet from the spinal catheter, introduced through a program of flow increases and height reductions, allow safe, high flow rates through the cerebral spinal pathway without undue intracranial pressure and without unduly low pressures in the cerebral spinal pathway.

### SUMMARY OF THE INVENTION

Provided, in one embodiment, is a method of delivering a physiologically acceptable liquid into the cerebral spinal pathway at high flow rates: initiating flow into a ventricular catheter, through a cerebral spinal pathway, and out a lumbar outflow catheter with the patient in a supine position and using a first flow rate, wherein a positive first value for an outlet pressure is maintained in plumbing from the lumbar outflow catheter; and increasing flow to a second flow rate greater than the first in conjunction with decreasing the outlet pressure to a second, negative value.

In another embodiment, the invention provides a method of delivering a physiologically acceptable liquid into at least a portion of the cerebral spinal pathway, collecting efflux of the physiologically acceptable liquid from the cerebral spinal pathway, and recycling the efflux, wherein the physiologically acceptable liquid is recycled by pumping with a first pump liquid into the cerebral spinal pathway and pumping with a second pump efflux for use in recycling, the method comprising operating one or both of the following safety procedures:

(a) operating the second pump at a flow rate target T2 higher than the operating target flow rate T1 of the first pump, and drawing gas into the efflux flow as needed to prevent the flow rate of the second pump from creating a significant negative pressure, wherein the higher rate of the second pump is adapted to prevent variance in the flow rates of the pumps or in compliance of a flow path from the first to the second pump from allowing the second pump to operate as a flow constrictor; or

(b) providing a bellows in an efflux flow pathway, lowering the T1:T2 ratio if the bellows expand or expand beyond an alarm-generating value, and increasing the T1:T2 ratio if the bellows contract or contract beyond an alarm-generating value.

### BRIEF DESCRIPTION OF THE DRAWINGS

FIGS. 1 and 2 display schematically a flow apparatus for flow through the cerebral spinal pathway.

FIG. 3 shows how the patient can be inclined during administration to achieve high flow rates.

FIG. 4 shows an efflux flow rate control mechanism.

FIG. 5 shows a pressure break device.

FIG. 6 illustrates another device for controlling pressure at the drainage end.

### DETAILED DESCRIPTION OF THE INVENTION

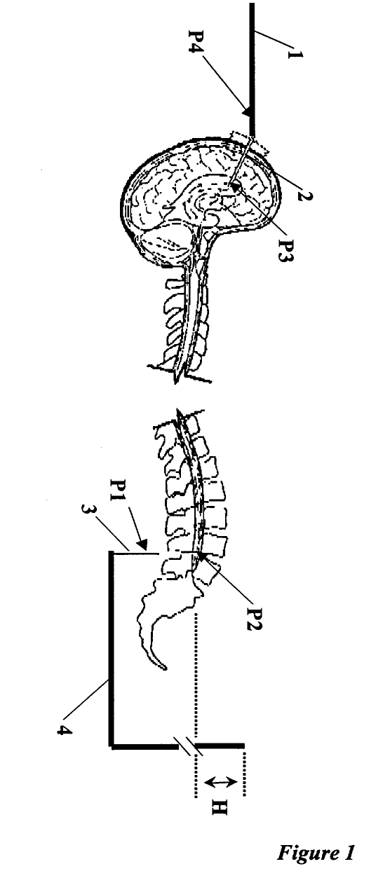

As illustrated in FIG. 1, treatment of a patient begins with the patient in a supine position. Tubing I delivers physiologically acceptable liquid (which can be solution, suspension or emulsion) to an ventricular catheter 2, positioned in the a lateral ventricle of the brain. Via the aqueduct, cisterna magna and subarachnoid spaces, a flow pathway is established to a lumbar outflow catheter 3, positioned for example at an intrathecal space of the lumbar (L4-L5) region of the spine. Any liquid that is physiologically acceptable for the central nervous system (CNS) can be used.

Pressure is monitored at the inlet to the cerebral spinal pathway, P4 (perfusion pressure, pressure at entrance to ventricular catheter), in an intracranial cavity, P3 (intracranial pressure, ICP), and at the outlet, Pt (drainage pressure, pressure at the exit of the lubar catheter). Pressure in the spinal cord can be measured at P2 (lumbar theca pressure), or that pressure can be inferred from other data and models based on past experience. All pressures are gage values. The outlet tubing 4 can have a spill-over set at a height H (column height) relative to a zero value that is aligned with the approximate center of the spinal column. H is illustrated as at a positive value, but negative values are used after flow rates have been ramped up.

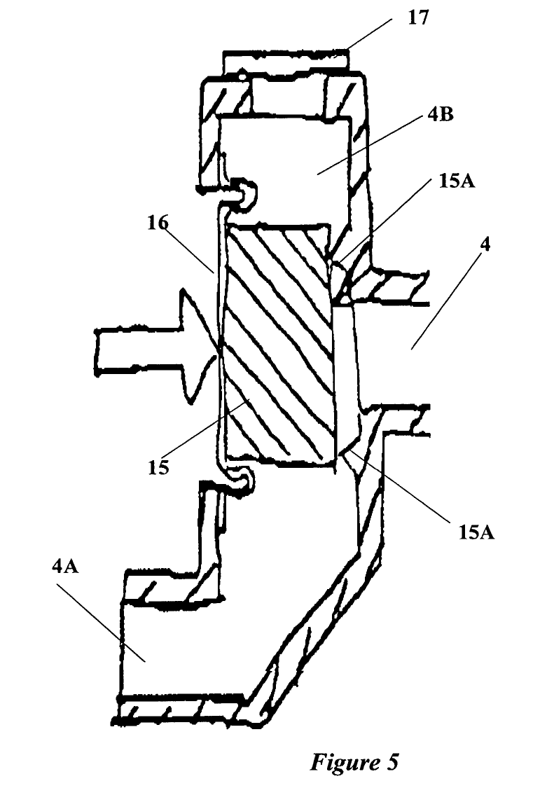

Height H is an illustrative way of setting the outlet pressure Pt. Other methods include for example using pressure break devices, actively controlling the input and output pump rates, and maintaining an expansion chamber (bellows) in the outlet tubing for which the expansion, and hence the pressure, can be actively managed. One illustrative pressure break device is illustrated in FIG. 5. Outlet tubing 4 is blocked, when the break pressure has not been obtained, by barrier piece 15, which seats on rim 15A. Rolled diaphragm 16 maintains liquid isolation. The break pressure is applied on the axis indicated by the arrow, and can be set by any of a number of mechanisms known in the art, such as spring-loaded tensioning devices, electromechanical pushing devices, hydraulic systems pressured by pumps or electromechanical pushing devices, gas pressure, and the like. In the illustrated break device, sterile filter 17 allows for gas (e.g., air) intake to manifold 4B, which is connected (independent of barrier piece 15) to outlet tubing 4A. To allow for negative pressure, the pressure break device can be positioned sufficiently below the H=0 level so that easing the break pressure effectively brings P1 to an appropriate negative value. Or, sufficient pumping can be applied to the fluid in outlet tubing 4A (in the absence of a gas intake) to maintain the desired negative pressure. Pressure control can be through active relative control of pumps (e.g., using the feedback loops and controller discussed below) or manual.

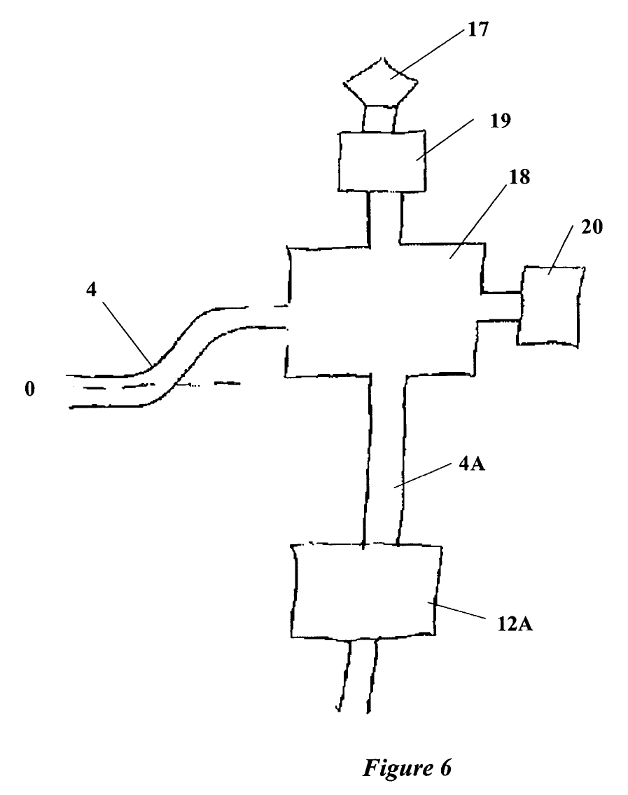

Another illustration of a pressure control device is found in FIG. 6. Manifold 18 is rigid, and can thus maintain a partial reduced pressure (measured against atmospheric). Manifold 18 is preferably placed above (e.g., 5 cm, measured from the bottom of the manifolds connection to tubing 4) the H=0 level. Valve 19 (if present) controls any introduction of gas into manifold 18, and can be for example a variable resistance valve or an adjustable pressure relief valve. Pressure monitor 20 can be a pressure transducer. Recycle pump 12A is suitable to create a reduced pressure in manifold 18. Preferably, pressure control is by active feedback control of recycle pump 12A, based on pressure data, for example from pressure monitor 20.

After initial setup of the catheters, the above introduced parameters can be, with no flow, for example:

| Body Position | H | P1 | P2 | P3 | P4 |
|---|---|---|---|---|---|
| Horizontal | +5 cm | 4 mmHg | 4 mmHg | 4 mmHg | 4 mmHg |

The values after initiation of flow are as set forth below for various flow rates. These values are based on the use of a 14 gauge catheter as the lumbar catheter. Exemplary catheters are described, for example, in co-pending Ser. No. 09/382,136, filed Nov. 26, 1999. The flow resistance of this catheter is a major determinant of P2, and consequently of P3. The use of catheters of different flow resistances will modify the pressure relationships as can be determined with appropriate calculations and modeling. When flow is at 10 mL/min:

| Body | H | P1 | P2 | P3 | P4 |
|---|---|---|---|---|---|
| Position | (cm) | (mmHg) | (mmHg) | (mmHg) | (mmHg) |
| Horizontal | +5 | 4 mmHg | 12 mmHg | 16.5 mmHg | 19.0 mmHg |
| Horizontal | 0 | 0 mmHg | 8 mmHg | 12.5 mmHg | 15.0 mmHg |
| Horizontal | −5 | −4 mmHg | 4 mmHg | 8.5 mmHg | 11.0 mmHg |

When flow is at 20 mL/min:

| Body | H | P1 | P2 | P3 | P4 |
|---|---|---|---|---|---|
| Position | (cm) | (mmHg) | (mmHg) | (mmHg) | (mmHg) |
| Horizontal | 0 | 0 | 16.75 | 27.0 | 32.25 |
| Horizontal | −10 | −8.0 | 8.75 | 19.0 | 24.25 |
| Horizontal | −15 | −12.0 | 4.75 | 15.0 | 20.25 |
| Horizontal | −20 | −16.0 | 0.75 | 11.0 | 16.25 |

When flow is at 30 mL/min:

| Body | H | P1 | P2 | P3 | P4 |
|---|---|---|---|---|---|
| Position | (cm) | (mmHg) | (mmHg) | (mmHg) | (mmHg) |
| Horizontal | −10 | −8.0 | 19.75 | 35.75 | 45.25 |
| Horizontal | −20 | −16.0 | 11.75 | 27.25 | 37.25 |
| Horizontal | −30 | −24.0 | 3.75 | 19.75 | 29.25 |

The shaded values are to be avoided. P2 values of less than about 3.5 are typically avoided.

The central nervous system (CNS) physiologically acceptable liquid used in the above example is a fluorocarbon nutrient emulsion containing eight a constituent compositions is as set forth in the table below for a 1200 mL unit of the emulsion. However, as mentioned, any CNS physiologically acceptable fluid can be used with this invention.

| | Amount |
|---|---|
| Constituent Compositions | g/unit |
| t-Bis-perfluorobutyl ethylene | 200 |
| NaCl, USP | 7.63 |
| NaHCO₃, USP | 2.19 |
| Purified egg yolk phospholipid, | 13.8 |
| KCl, USP | 0.23 |
| MgCl₂—6H₂O, USP | 0.24 |
| CaCl₂—2H₂O, USP | 0.18 |
| Dextrose, USP | 1 |
| Albumin (Human), USP | 20 |
| L-lysine HCl, USP | 0.0032 |
| L-alanine, USP | 0.0034 |
| L-serine, USP | 0.0030 |
| L-threonine, USP | 0.0036 |
| L-arginine, USP | 0.0022 |
| L-leucine, USP | 0.0015 |
| L-isoleucine, USP | 0.0006 |
| L-valine, USP | 0.0020 |
| L-phenylalanine, USP | 0.0010 |
| L-tyrosine, USP | 0.0010 |
| L-histidine, USP | 0.0012 |
| L-methionine, USP | 0.0003 |
| NaH₂PO₄, USP | 4.1 |
| Na₂HPO₄, USP | 0.61 |
| α-ketoglutaric acid | 0.030 |
| Sterile Water for Injection, USP | 1040 mL |

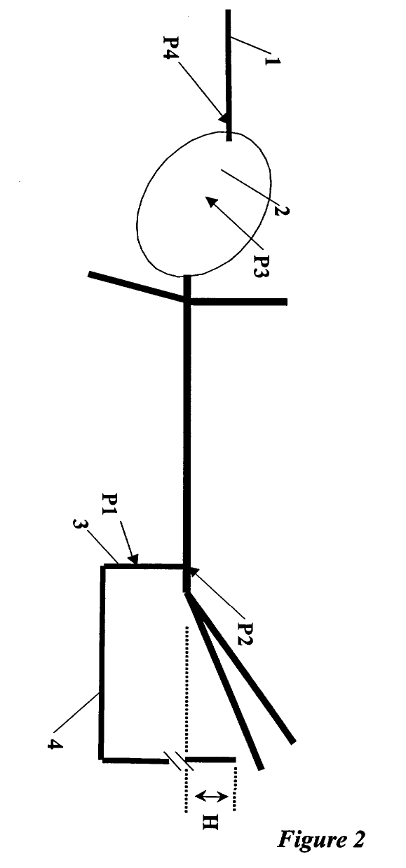

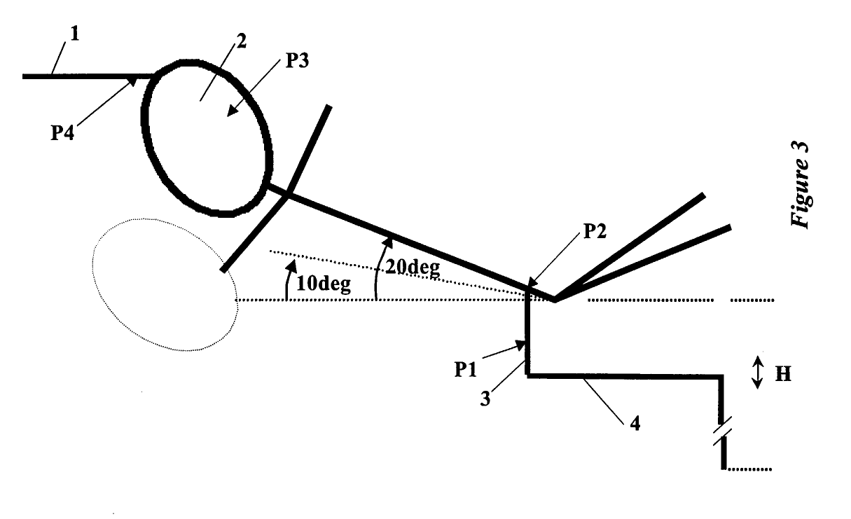

FIG. 2 shows elements of FIG. 1 in a more schematic fashion. After higher flow has been initiated, the patient can be elevated as indicated in FIG. 3. For example, the patient can be safely inclined when flow rates have become high, such as 20 mL/min, 25 mL/min, 30 mL/min or higher. In FIG. 3, the patient is illustrated at a 20 degree incline, with a 10 degree incline illustrated in dotted lines. Incline angles are often in the lower range of, for example, 10 or 20 degrees, but higher inclinations can be used to achieve still more elevated flow rates, such as 50, 60 or 70 mL/min.

Exemplary pressure parameters with an incline are illustrated below. Body position only affects ICP (P3) and perfusion pressure (P4), lumbar theca (P2) and drainage (P1) pressures are unaffected. A five degree incline will reduce ICP and PP by 3.75 mmHg for an average sized patient, by 7.25 mmHg for a 10 degree incline, by 11.0-mmHg for a fifteen degree incline, and by 14.50 mmHg for a twenty degree incline. For example, when flow is at 30 mL/min:****

| Body | H | P1 | P2 | P3 | P4 |
|---|---|---|---|---|---|
| Position | (cm) | (mmHg) | (mmHg) | (mmHg) | (mmHg) |
| Horizontal | −30 | −24.0 | 3.75 | 19.8 | 29.3 |
| 5° incline | −30 | −24.0 | 3.75 | 16.0 | 25.5 |
| 10° incline | −30 | −24.0 | 3.75 | 12.5 | 22.0 |

In another aspect, the delivery algorithm takes into account a phenomenon (and risk) involved in recycling the liquid that has cycled through the cerebral spinal pathway back through the pathway. A mismatch in inflow and outflow rates can occur, resulting from the tolerances in the two pumping systems, a difference in CSF production and absorption, or a change in ICP and the concurrent change in CNS volume due to compliance in the CNS. Such a mismatch could lead to an over or under pressure condition in the patient. This risk is addressed in one or more of two ways.

First, the flow rate of pumping of the effluent is maintained a rate sufficiently higher than the delivery flow rate to account for these sources of variation. An gas/air intake (preferably fitted with a sterile filter) in the effluent line provides a fluid source to account for the higher flow rate. The intake is linked to the tubing/plumbing before the pump inlet. Before recycle, the liquid can be passed through a holding container in which the extra gas is separated away (preferably through a sterile filter). This format is effective to not, of itself, create a significant negative pressure. The minor pressure differential across the sterile filter is not a significant pressure. A device for accomplishing these functions with peristaltic pumps is described in U.S. Ser. No. 60/286,057, filed Apr. 24, 2001 and its successor application U.S. Ser. No. ______ filed concurrently herewith. These applications are incorporated by reference herein in their entirety. A preferred set-point in the flow differential is between 5 and 15%, such as about 10%. Note, that this is the differential set with respect to the average calibrated flow rate, but in some instances the differential is established in part in acknowledgement that the pumps used for reliable, non-pulsatile, sterile pumping can be somewhat variable in their actual flow rate.

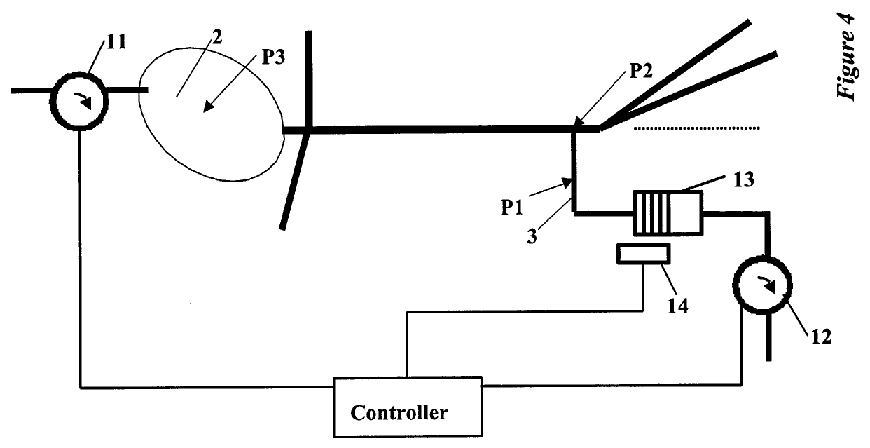

Second, as illustrated in FIG. 4, a bellows 13 is incorporated into the tubing/plumbing before the recycle pump 12, and the expansion or contraction of the bellows is monitored by monitor 14. Monitor 14 sends data to the controller, which adjusts the rate of delivery pump 11 or recycle pump 12 as appropriate. Data from pressure monitoring devices can also be sent to the controller, so that the controller can avoid increasing the flow of delivery pump 11, or reduce the flow of delivery pump 11, in response to pressure data.

The monitor 14 can be physically connected to the bellows via a linear transducer or linear potentiometer, providing an electrical signal for the amount of movement in the bellows. Or, the monitor can monitor the offset of the below with a light reflectance angle, with multiple reflectance monitors that indicate whether the bellows is within or without a reflectance pathway, by measuring the distance analog of an acoustic reflectance. Other methods recognized in the art for measuring displacements can be used. Where a bellows such as illustrated functions to control pressure at the drainage end, the same devices for controllably applying pressure as discussed above with reference to the break pressure can be used to exert the required force on the bellows.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A method of delivering a physiologically acceptable liquid into the cerebral spinal pathway:
initiating flow into a ventricular catheter, through a cerebral spinal pathway, and out a lumbar outflow catheter with the patient in a supine position and using a first flow rate, wherein a positive first value for an outlet pressure is maintained in plumbing from the lumbar outflow catheter; and
increasing flow to a second flow rate greater than the first in conjunction with decreasing the outlet pressure to a second, negative value.

2. The method of claim 1, further comprising:
at some point after finishing the flow at the first rate, elevating the patient.

3. The method of claim 1, further comprising:
between initiating flow to the first rate and increasing to the second rate, adjusting the flow rate to an intermediate rate between the first and second rate in conjunction with adjusting the outlet pressure to a value between the first and second value.

4. The method of claim 3, further comprising:
at some point after finishing the flow at the first rate, inclining the patient.

5. The method of claim 1, wherein the second flow rate is 20 mL/min or higher.

6. The method of claim 1, wherein the physiologically acceptable liquid is recycled through the cerebral spinal pathway by pumping with a first pump liquid into the cerebral spinal pathway and pumping with a second pump efflux from the cerebral spinal pathway for use in recycling, wherein the method further comprises operating one or both of the following safety procedures:
(a)
operating the second pump at a flow rate target T2 higher than the operating target flow rate T1 of the first pump, and
drawing gas into the efflux flow as needed to prevent the flow rate of the second pump from creating a significant negative pressure, wherein the higher rate of the second pump is adapted to prevent variance in the flow rates of the pumps or in compliance of a flow path from the first to the second pump from allowing the second pump to operate as a flow constrictor; or
(b)
providing a bellows in an efflux flow pathway,
lowering the T1:T2 ratio if the bellows expand or expand beyond an alarm-generating value, and
increasing the T1:T2 ratio if the bellows contract or contract beyond an alarm-generating value.

7. A method of delivering a physiologically acceptable liquid into at least a portion of the cerebral spinal pathway, collecting efflux of the physiologically acceptable liquid from the cerebral spinal pathway, and recycling the efflux, wherein the physiologically acceptable liquid is recycled by pumping with a first pump liquid into the cerebral spinal pathway and pumping with a second pump efflux for use in recycling, the method comprising operating one or both of the following safety procedures:
(a)
operating the second pump at a flow rate target T2 higher than the operating target flow rate T1 of the first pump, and
drawing gas into the efflux flow as needed prevent the flow rate of the second pump from creating a significant negative pressure, wherein the higher rate of the second pump is adapted to prevent variance in the flow rates of the pumps or in compliance of a flow path from the first to the second pump from allowing the second pump to operate as a flow constrictor, or
(b)
providing a bellows in an efflux flow pathway,
lowering the T1:T2 ratio if the bellows expand or expand beyond an alarm-generating value, and
increasing the T1:T2 ratio if the bellows contract or contract beyond an alarm-generating value.

8. The method of claim 7, comprising further to (a):
returning efflux to a location within a container above a level of liquid resident therein,
bleeding excess gas volume from the container through a sterile filter, and,
drawing liquid from the container for pumping into the cerebral spinal pathway.

9. The method of claim 7, according to (a), wherein T2 is at least 5% higher than T1.

10. The method of claim 7, according to (a), wherein T2 is at least 10% higher than T1.
